(19) United States
(12) Patent Application Publication
(10) Pub. No.: US 2006/0079746 A1
(43) Pub. Date: Apr. 13, 2006

(54) Apparatus and method for analysis of tissue classes along tubular structures

(76) Inventors: Florence Marie-Francoise Perret, Paris (FR); Laurent Launay, Saint Remy les Chevreuse (FR); Melissa L. Vass, Milwaukee, WI (US); Kelly Ann Mohr, New Berlin, WI (US); Amy L. Broadie, Milwaukee, WI (US)

Correspondence Address: CANTOR COLBURN, LLP, 55 GRIFFIN ROAD SOUTH, BLOOMFIELD, CT, 06002, US

(21) Appl. No.: 11/039,591

(22) Filed: Jan. 20, 2005

Related U.S. Application Data
(60) Provisional application No. 60/617,872, filed on Oct. 11, 2004.

(51) Int. Cl. A61B 5/05

(52) U.S. Cl. 600/407

(57) ABSTRACT

A method for analyzing tissue classes along a tubular structure defined by voxels is disclosed. A tubular shaped region of interest (ROI) is constructed along a predetermined centerline of the tubular structure according to the following: at least one point is defined along the centerline to define the extremities of the ROI; a diameter corresponding to the ROI is defined and/or computed; contiguous unit volumes are applied along the centerline between the extremities of the ROI; a first volume is computed by the union of the unit volumes; and, a final volume of the ROI is defined as the connex part of the first volume that contains the middle of the tubular structure. The final volume is then analyzed with respect to tissue classes present therein.

### CROSS-REFERENCE TO RELATED APPLICATIONS

This application claims the benefit of U.S. Provisional Application Ser. No. 60/617,872, entitled “Apparatus and Method for Analysis of Tissue Classes Along Tubular Structures”, filed Oct. 11, 2004, which is incorporated herein by reference in its entirety.

### BACKGROUND OF THE INVENTION

The present disclosure relates generally to an apparatus and method for analyzing tissue classes along a tubular structure, and particularly for analyzing the degree of plaque burden in a vessel.

Extracting vessels from a 3D medical image is very important to help diagnostic tasks. There is a market today for visualizing and quantifying calcified plaque deposits in the vessels from non-contrast Computed Tomography (CT) examinations. Improved resolution provided by today's imaging systems, however, is bringing physicians closer to seeing various levels of soft plaque deposits in the vessels in addition to the high density calcified plaque. Assessment of soft plaque has increasing clinical value as we are able to understand a larger number of the risks related to the deposits (soft plaque is more likely to break loose in the blood stream and cause stroke, for example). In view of the capabilities and limitations of today's CT post processing systems, post processing vessel analysis is becoming a more time consuming step to reach diagnosis. Vessel tracking software exists today (Automatic Vessel Tracking Analysis, AVA) to help focus the data of interest in a single viewport instead of requiring the reviewer to page through full axial image series upfront. Targeting areas of increased hard and soft plaque deposit along the vessel would assist in the overall exam assessment as well by highlighting areas that may be missed by the human eye. In addition to providing an exam layout for review that helps to direct the exam analysis for the physicians, any steps provided to help automate or provide a fast 3D review for the initial read help to increase the findings and clarity of information found in the review process. Segmentation has been developed to isolate areas of interest and create 3D volume models of specified anatomy, but additional methods are needed to visualize characteristics of the anatomical wall and inside the area of interest by a quick review.

Quantitative classification and volume measurement tools do not currently track and visually display results along a tracked vessel. Accordingly, there is a need in the art for an apparatus and method for the analysis of tissue classes along tubular structures that overcomes these drawbacks.

### BRIEF DESCRIPTION OF THE INVENTION

Embodiments of the invention include a method for analyzing tissue classes along a tubular structure defined by voxels. A tubular shaped region of interest (ROI) is constructed along a predetermined centerline of the tubular structure according to the following: at least one point is defined along the centerline to define the extremities of the ROI; a diameter corresponding to the maximum of the diameters of the orthogonal sections of the ROI is defined and/or computed; a plurality of contiguous unit volumes are applied along the centerline between the extremities of the ROI, the unit volumes having an overall dimension equal to or less than the maximum of the diameters of the orthogonal sections of the ROI; a first volume is computed by the union of the unit volumes; and, a final volume of the ROI is defined as the connex part of the first volume that contains the middle of the tubular structure. The final volume is then analyzed with respect to tissue classes present therein.

Other embodiments of the invention include an apparatus for acquiring tissue images and analyzing tissue classes along tubular structures. The apparatus includes a medical scanner for generating a volume of image data relating to a region of interest, a data acquisition system for acquiring the volume of image data, an image reconstructor for reconstructing a viewable image from the volume of image data, a database for storing information from the data acquisition system and the image reconstructor, an operator interface for managing the medical scanner, the data acquisition system, the image reconstructor, the database, or any combination thereof, a computer for analyzing the reconstructed volume of image data and displaying the viewable image, the computer being responsive to said operator interface, and a storage medium, readable by a processing circuit, storing instructions for execution by the processing circuit for practicing embodiments of the aforementioned method.

Further embodiments of the invention include a computer program product embodied in a tangible medium for analyzing tissue classes along tubular structures. The product includes computer readable instructions for practicing embodiments of the aforementioned method.

### BRIEF DESCRIPTION OF THE DRAWINGS

Referring to the exemplary drawings wherein like elements are numbered alike in the accompanying Figures:

FIGS. 1A and B depict exemplary images of a coronary vessel tracked and displayed in the lumen view and the curved reformat plane, respectively;

FIG. 2 depicts a generalized pictorial view of a CT imaging system for acquiring and analyzing image data from a patient in accordance with embodiments of the invention;

FIG. 3 depicts a generalized block schematic diagram of the imaging system of FIG. 2;

FIG. 4 depicts an exemplary method of building a region of interest (ROI) along the centerline of a vessel in accordance with embodiments of the invention;

FIG. 5 depicts an exemplary visual-coding scheme relative to Hounsfield Unit (HU) ranges for use in accordance with embodiments of the invention;

FIGS. 6A, B, C and D depict exemplary images of a vessel in curved reformat view with and without visual coding applied in accordance with embodiments of the invention;

FIG. 7 depicts an exemplary table of quantitative output with respect to the image depicted in FIG. 6D;

FIG. 8 depicts another exemplary table, similar to that of FIG. 5, but of quantitative output with respect to the two regions;

FIG. 9 depicts an exemplary image illustrating the two regions relating to the table of FIG. 8;

FIGS. 10A, B and C depict exemplary images in oblique, best L, and cross-section view, respectively, in accordance with embodiments of the invention; and

FIGS. 11A and B depict exemplary images with continuous mode off and continuous mode on, respectively, in accordance with embodiments of the invention.

### DETAILED DESCRIPTION OF THE INVENTION

An embodiment of the invention provides a visual coding for vessel analysis that allows the user to identify a specific length along a tracked vessel and view that specific region in visual coding instead of the standard grayscale typically used for reviewing computed tomography (CT) exams generally. While embodiments of the invention are described with reference to CT scanning, it will be appreciated that the scope of the invention as herein disclosed is not necessarily limited to a single modality of medical analysis, and may be applied to any modality of medical analysis capable of reproducing images of medical anatomy that may then be visually coded by techniques herein disclosed. Exemplary visual coding includes color coding, cross-hatch coding, speckle-density coding, or any other pattern coding that visually distinguishes one region from another, and a region of interest from surrounding tissue displayed in grayscale. This capability allows the user to get quantitative and qualitative information to quickly assess the vessel such as, for example, by identifying type and degree of plaque burden in the vessel, contrast flow through a stent, or plaque outside the vessel wall. These details may help to direct the exam review and improve workflow and results seen from CT exams.

Visual-coding of a specific region in vessel analysis provides the user with a method for quick assessment and analysis of various tissue classes and various tissue densities in and along a vessel. Embodiments of the invention may be applied to any tracked vessel including but not limited to Carotid, Coronary Sinus, and coronary arteries. For exemplary purposes only, coronary vessels will be used in the following description and figures. Embodiments of the invention are composed of two different parts: construction of a tubular region of interest, and analysis of the tissues among this volume.

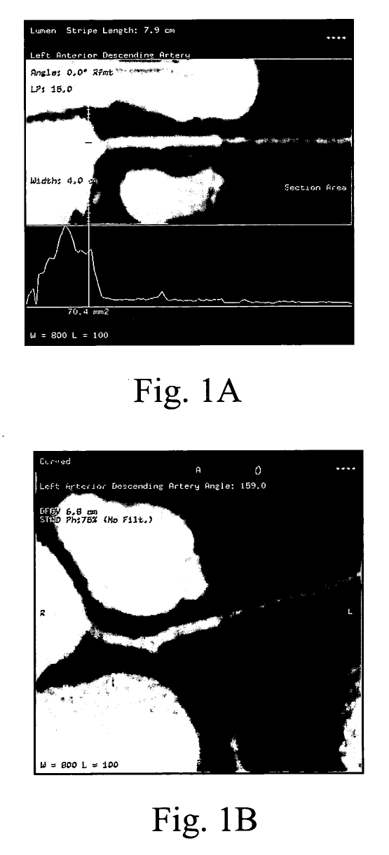

As a precursor to embodiments of the invention, a vessel tracking software, such as Automatic Vessel Tracking Analysis (AVA) as disclosed in U.S. Pat. No. 6,718,193 commonly assigned, for example, is applied to create a centerline down the length of a vessel (such as a coronary vessel for example), which allows the vessel to be viewed in multiple layouts, such as in lumen view (stretching the vessel out straight in a plane to assess the diameter etc, see FIG. 1A) and a curved reformat view (laying the curved vessel all in a single plane with surrounding tissue distorted out of plane, see FIG. 1B). As a general matter, FIGS. 1A and 1B depict computed tomography (CT) images defined by voxels of varying intensity according to the Hounsfield Unit (HU) scale. In accordance with embodiments of the invention, once the centerline of the vessel is established, a region of interest (ROI) is established and visual-coding is applied to the ROI along the centerline of the tracked vessel.

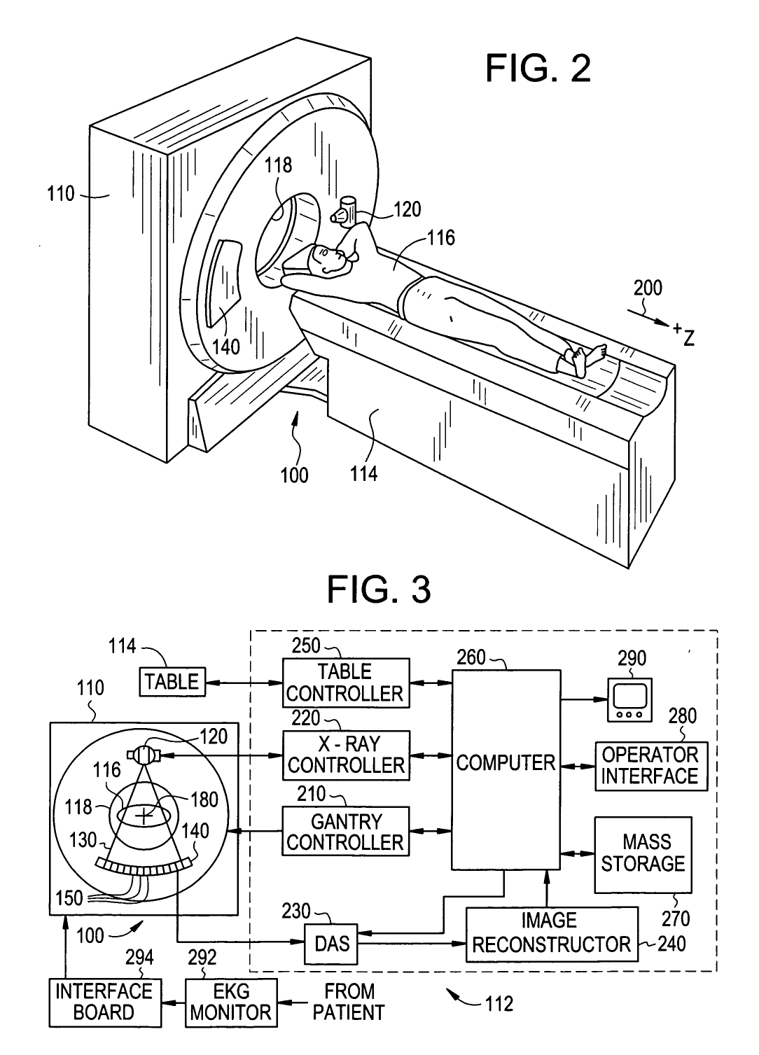

In an exemplary embodiment, the images of FIGS. 1A and 1B are generated using the imaging system 100 depicted in FIGS. 2 and 3, which in an exemplary embodiment employs cardiac imaging by computed tomography (CT). However, embodiments of the invention are applicable to all relevant cardiac imaging modalities including, but not limited to CT, magnetic resonance imaging, radionuclide imaging, echocardiography (Ultrasound), positron emission tomography (PET).

Referring to FIGS. 2 and 3, a computed tomography (CT) imaging system 100 is shown having a gantry 110, which is representative of a CT scanner (scanner), a control system 112, and a motorized table 114 for positioning an object 116, such as a patient, in gantry opening 118 in gantry 110. Gantry 110 includes an x-ray source 120 that projects a fan beam of x-rays 130 toward a detector array 140 on the opposite side of gantry 110. Detector array 140 is formed by detector elements 150, which may include a single row or multiple rows of elements 150. Detector elements 150 are radiation detectors that each produce a signal having a magnitude that represents and is dependent on the intensity of the attenuated x-ray beam 130 after it has passed through patient 116 being imaged. During a helical scan that acquires x-ray projection data, the gantry 110 along with the x-ray source 120 and detector array 140 rotate within the imaging plane and around the patient 116 about a center of rotation 180, while the patient 116 is moved through the gantry in a z-direction 200 perpendicular to the imaging plane.

Gantry 110 and x-ray source 120 are controlled by control system 112, which includes a gantry controller 210, an x-ray controller 220, a data acquisition system (DAS) 230, an image reconstructor 240, a table controller 250, a computer 260, a mass storage (database) system 270, an operator interface 280, and a display device 290. Gantry controller 210 controls the rotational speed and position of gantry 110, x-ray controller 220 provides power and timing signals to x-ray source 120, data acquisition system 220 acquires analog data from detector elements 150 and converts the data to digital form for subsequent processing, image reconstructor 240 receives the digitized x-ray data from DAS 230 and performs an image reconstruction process for subsequent cardiac analysis, as discussed below, and table controller 250 controls motorized table 114 to position patient 116 in gantry opening 118.

Computer 260 is in operable communication with gantry controller 210, x-ray controller 220, and table controller 250 whereby control signals are sent from the computer to controllers 210, 220, 250 and information is received from the controllers by computer 260. Computer 260 also provides commands and operational parameters to DAS 230 and receives reconstructed image data from image reconstructor 240. In an alternative embodiment, DAS 230 and image reconstructor 240 may be integrated with computer 260. The reconstructed image data is stored by computer 260 in a mass storage device 270 for subsequent retrieval. An operator interfaces with computer 260 through operator interface 280, which may include, for example, a keyboard and a graphical pointing device, and receives output, such as, for example, a reconstructed image, control settings and other information, on a display device 290.

Operable communication between the various system elements of FIG. 1 is depicted by arrowhead lines, which illustrate a means for either signal communication or mechanical operation, depending on the system element involved. Operable communication amongst and between the various system elements may be obtained through a hardwired or a wireless arrangement. Computer 260 may be a standalone computer or a network computer and may include instructions in a variety of computer languages for use on a variety of computer platforms, such as, for example, DOS™-based systems, Apple™-based systems, Windows™-based systems, HTML-based systems, or the like.

CT imaging system 100 includes an electrocardiogram (EKG) monitor 292 that outputs R-peak events, which generally delineate the beginning of a heart cycle. The EKG monitor 292 is coupled to scanner 110 through an interface board 294 and enables synchronization between the scanner data and the EKG monitor data. Alternatively, the interface board 294 may be used to couple the EKG monitor 292 to scanner 110. An example of an interface board 294 is a Gantry interface board. The exemplary scanner 110 is a cardiac computed tomography (CT) system with support for cardiac imaging, however, the illustrated scanner 110 is for exemplary purposes only; other imaging systems known in the art may also be used. Examples of other imaging systems include, but are not limited to, X-ray systems (including both conventional and digital or digitized imaging systems), magnetic resonance (MR) systems, positron emission tomography (PET) systems, ultrasound systems, nuclear medicine systems, and 3D fluoroscopy systems. CT imaging system 100 also includes EKG gated acquisition or image reconstruction capabilities to image the heart free of motion artifact, typically in its diastolic phase for optimum image quality. CT imaging system 100 further includes circuitry for acquiring image data at DAS 230 where the data is transformed into a useable form and processed at image reconstructor 240 to create a reconstructed image of features of interest within the patient. The image data acquisition and processing circuitry is often referred to as a “scanner”, regardless of the type of imaging system, because some sort of physical or electronic scanning often occurs in the imaging process. The particular components of the system and related circuitry differ greatly between imaging systems due to the different physics and data processing requirements of the different system. However, it will be appreciated that the present invention can be applied regardless of the selection of a particular imaging system.

Data are output from scanner 110 into control system 112 that includes software to perform data acquisition in data acquisition system 230, and image generation in image reconstructor 240. Data control is provided by operator interface 280. Data that is output from the scanner 110 is stored in mass storage 270. Data acquisition is performed according to one or more acquisition protocols that are optimized for imaging the heart, and specifically for imaging the left ventricle and myocardial muscle. Image generation in image reconstructor 240 is performed using one or more optimized 3D protocols for automated post-processing of the CT image dataset.

Computer 260 includes known visualization algorithms for use with medical CT imaging data, such as, for example, multiplanar volume reformat (MPVR), Maximum Intensity Projection (MIP), 3D surface rendering or volume rendering (VR), immersible viewing (i.e., viewing from the inside), and Automatic Vessel Tracking Analysis (AVA), which may be used for detecting vessel stenosis. A variety of 3D software packages for volume analysis and cardiac image quality analysis are also available.

Exemplary embodiments of the invention may employ the aforementioned programs on computer 260 for the acquisition and post-processing of cardiac data relating to coronary artery disease, acute cardiac syndromes, coronary artery imaging, cardiac function analysis, myocardial perfusion analysis, myocardial perfusion defect analysis, automated left ventricle delineation, automated volume rendering, automated cardiac phase selection, end diastole volume analysis, end systole volume analysis, stroke volume analysis, ejection fraction analysis, and cardiac output analysis, all from a single cardiac CT scan.

Embodiments of the invention also include the aforementioned visual coding scheme that is applied to the region of interest (ROI) along the centerline of the tracked vessel, which will now be discussed in more detail.

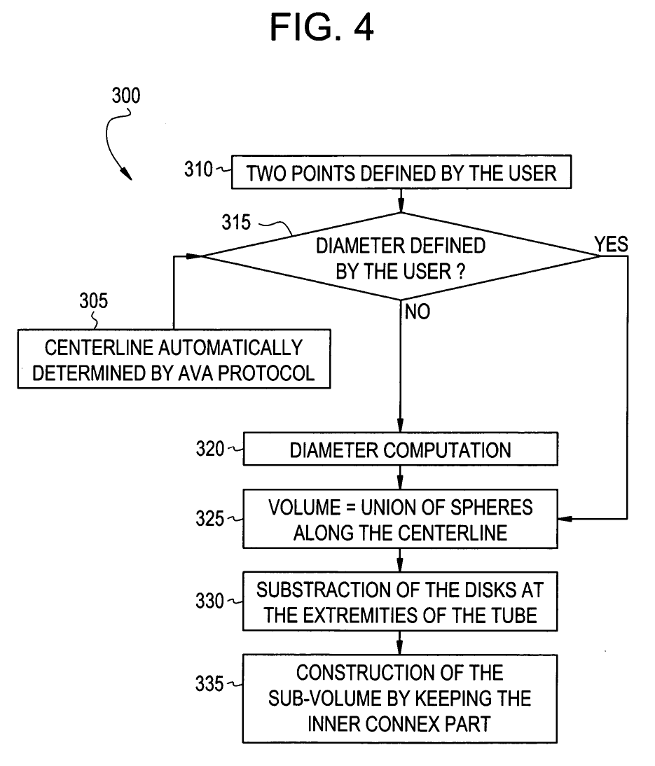

Referring now to FIG. 4, the ROI is established by constructing a sub-volume tube along the centerline of the vessel, which is more generally herein referred to as a tubular structure. As a general matter, the ROI is that portion of the tubular structure that corresponds to the plaque that the user wants to analyze.

The method 300 of FIG. 4 has as an input 305 a predetermined centerline of the tubular structure that has been automatically determined, or predefined, by the aforementioned vessel tracking analysis software.

At block 310, the user defines two points along the centerline of the tubular structure to define the extremities of the ROI, or more generally defines at least one point from which the ROI may grow. To establish a diameter about the centerline for the ROI, the user has the choice of either manually defining the diameter (block 315), or permitting the aforementioned vessel tracking analysis software to automatically compute the diameter (block 320). In an embodiment, the diameter of the ROI between the extremities corresponds to the maximum of the diameters of the orthogonal sections of the ROI. However, between the extremities, the diameter of the ROI may be variable and adjustable, thereby enabling the user to view plaque formations that grow and shrink in overall diameter along the ROI.

At block 325, a plurality of contiguous unit volumes, such as spheres, cylinders or any set of pre-defined 3D volume elements, is applied along the centerline between the extremities of the ROI, and then joined to define a first volume by the union of the unit volumes. Each unit volume has an overall dimension equal to or less than the maximum diameter of the associated orthogonal section of the ROI.

At block 330, the extremities of the first volume are optionally modified by subtracting two other volumes, one from each extremity, to establish flat surfaces at the extremities of the first volume. This optional procedure may be implemented for statistical analysis using high resolution CT imaging.

At block 335, the final volume of the ROI is computed by that volume of the modified first volume defined by the connex part that contains the middle of the tubular structure.

At the conclusion of method 300, a tubular region of interest is available for analysis.

While FIG. 4 depicts one method of computing a volume of the ROI, the volume may also be computed by other techniques, such as dilation of the centerline of the vessel or burning of voxels whose distance to the centerline is less than the diameter, for example.

Upon computing the volume of the ROI, the user may then adjust parameters such as the length of the volume (the start and end points, or extremities), or the diameter of the volume, thereby being able to adjust the volume around the specific ROI.

Once the ROI is built, different tools may be used to analyze its content. A visual coding scheme that uses a Look-Up-Table (LUT) approach, for example, may be applied to the ROI. Visual-coding works by applying a set distinguishing visuals, such as colors or patterns, to each neighborhood of voxels within a set range according to the Hounsfield Unit (HU, basic CT unit of measurement) of that voxel. Default settings, for example, could be used to classify the visually coded region into the following four ranges of HU values:

20-60

60-150

150-350

350-1000,

where the user may specify a distinguishing visual and a name for each HU range, such as, for example,

| HU Range | Visual | Name |
|---|---|---|
| 20-60 | blue, or low density speckle shading | soft plaque |
| 60-150 | yellow, or high density speckle shading | fibrous plaque |
| 150-350 | green, or single cross-hatch shading | fibrocalcified |
| | | plaque |
| 350-1000 | red, or double cross-hatch shading | calcified plaque. |

While specific HU ranges are given above, it will be appreciated that this is for illustration purposes only, and that the user may define alternative (user defined) HU ranges. While the specified name makes reference to a plaque formation, such as soft, fibrous, fibrocalcified, or calcified, it will be appreciated that this is for illustration purposes only, and that the user may utilize alternative characterizations, such as vessel deposits of varying density for example.

Accordingly, the user could customize the number of ranges, the maximum and minimum values of each range, the distinguishing visual of each range, and the name/label for each range. These values may be saved and modified in a Look-Up Table (LUT). A benefit of the LUT is to enable the separation of the different pixels or voxels into different distinguishing visual classes in order to be able to calculate some volumetric percentages corresponding to the different tissues.

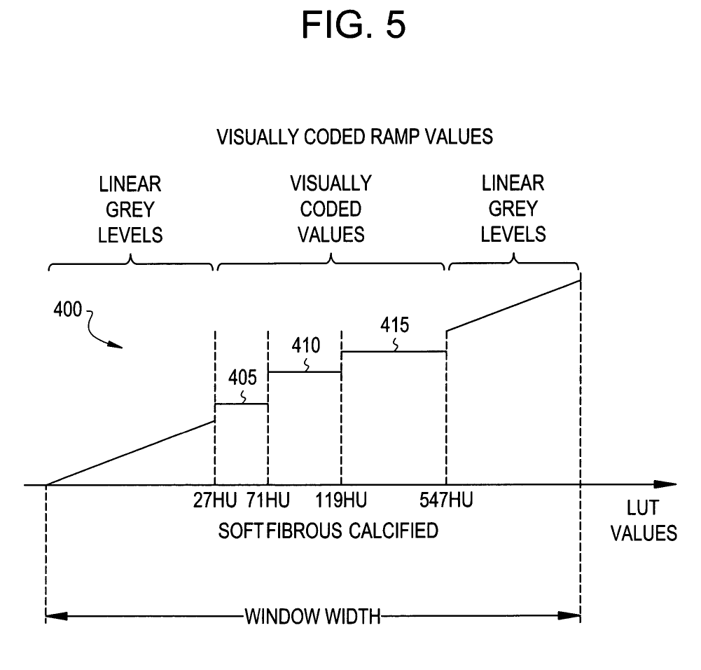

FIG. 5 depicts an alternative illustration of how the user may define distinguishing visuals with respect to visual coding in three different HU ranges using a look up table (LUT) 400. In FIG. 5, a vessel deposit representative of a soft plaque has an HU range of 27-71 (denoted by numeral 405) and may be visually coded by the color blue or by speckling, a vessel deposit representative of a fibrous plaque has an HU range of 71-119 (denoted by numeral 410) and may be visually coded by the color yellow or by single cross-hatching, and a vessel deposit representative of a calcified plaque has an HU range of 119-547 (denoted by numeral 415) and may be visually coded by the color red or by double cross-hatching. As can be appreciated, the user may define how the LUT 400 is constructed.

Qualitatively the visually coded feature may also allow the user to modify the number of regions or length of the existing regions, as well as the ability to insert an intermediate region that is void of a visual coding (left as a standard gray scale image). This capability could be very useful in the case of a stent implanted in a vessel, for example, where the doctor may want to create regions such that the KU range covering the stent itself is not visually coded and not taken into account in the volumetric measurements. Similarly, on a standard vessel, the user could choose to remove visual coding from the HU range representing the lumen and visually code only the wall and the area of a specified diameter from the centerline.

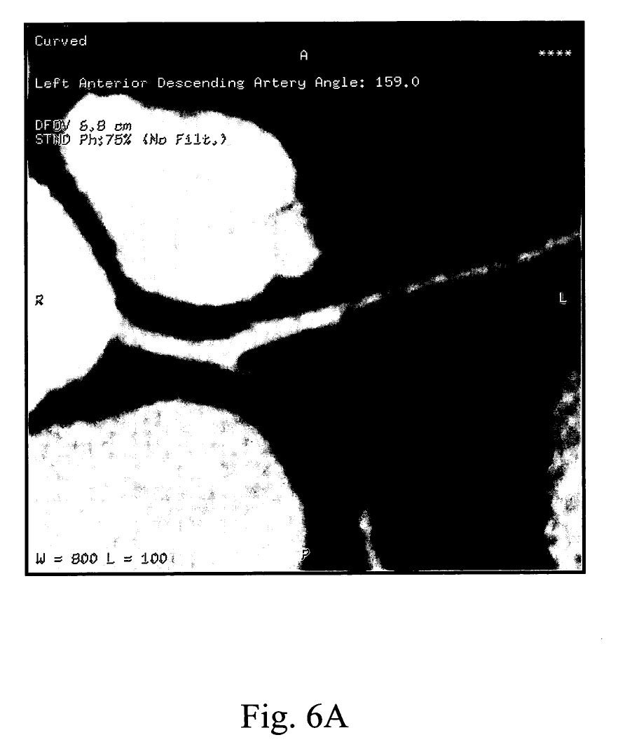

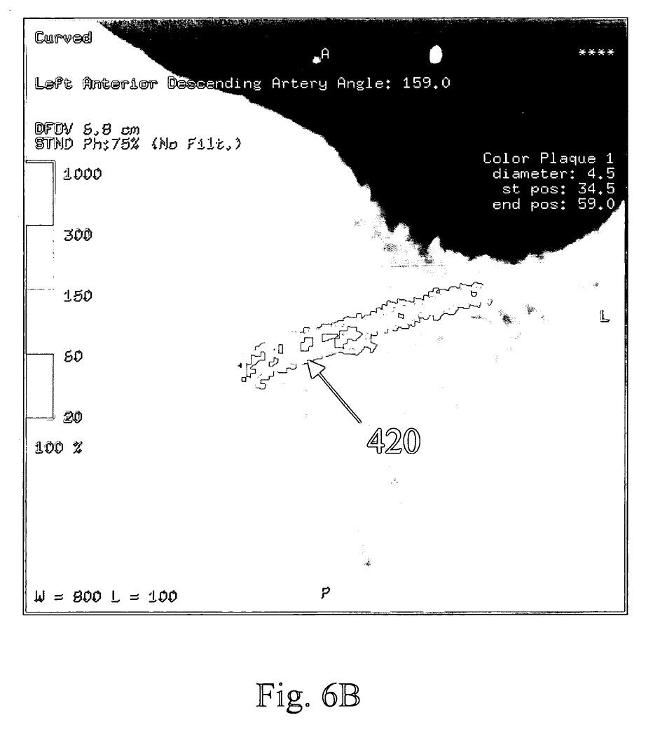

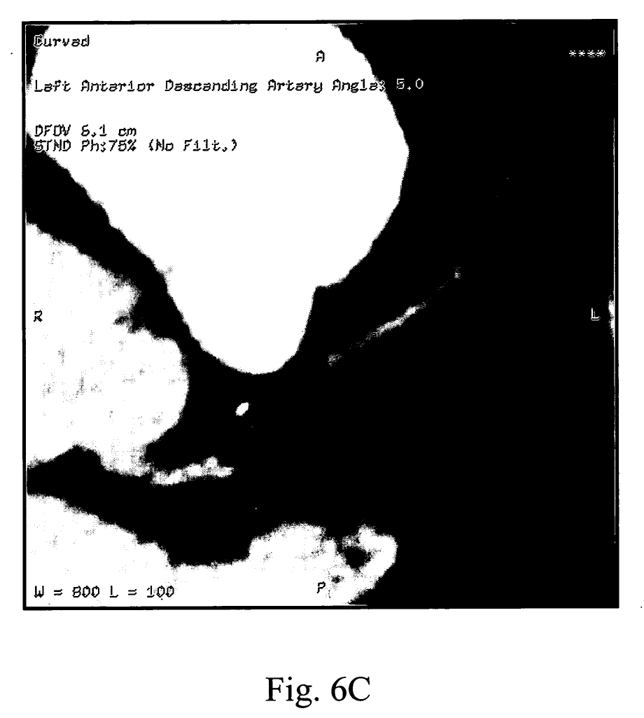

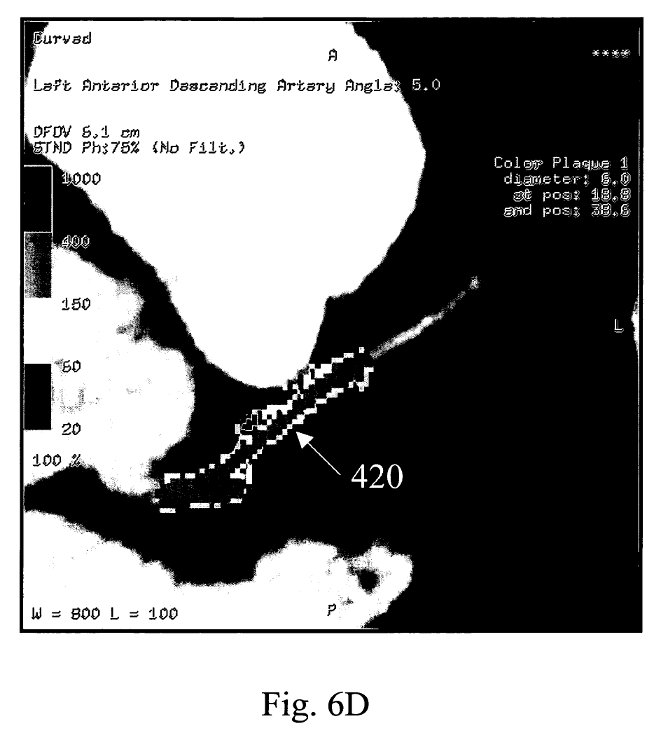

Referring now to FIGS. 6A, B, C and D, which depict images of a vessel in curved reformat view, a user may analyze a visually coded ROI with respect to a classification of tissue densities associated with the visual coding (denoted by numeral 420 in FIGS. 6B and 6D), with each tissue density classification having a density range correlating with a visual coding. In FIGS. 6A, B, C and D, for example, a proximal region of the Left Anterior Descending coronary vessel having a plaque burden of unknown type is depicted without visual coding in FIGS. 6A and C, and with visual coding in FIGS. 6B and D. A fibrous cap is depicted by double cross-hatch shading (see FIG. 6D). Here, alternative to the LUT of FIG. 5, visual coding is provided in four different HU ranges.

By using visual coding, the ROI may be analyzed both visually and mathematically with respect to the volume of each density range, or with respect to a statistical analysis of each density range. Quantitatively, embodiments of the invention may then provide a volume of each specific range as well as an overall volume of the visually coded region. In an exemplary ROI, quantitative outputs may be, for example:

20-60, blue (low density speckling), soft plaque, 0.45 mm³

60-150, yellow (high density speckling), fibrous plaque, 0.80 mm³

150-350, green (single cross-hatch), fibrocalcified plaque, 0.55 mm³

350-1000, red (double cross-hatch), calcified plaque, 0.20 mm³

Total Volume=2.0 mm³.

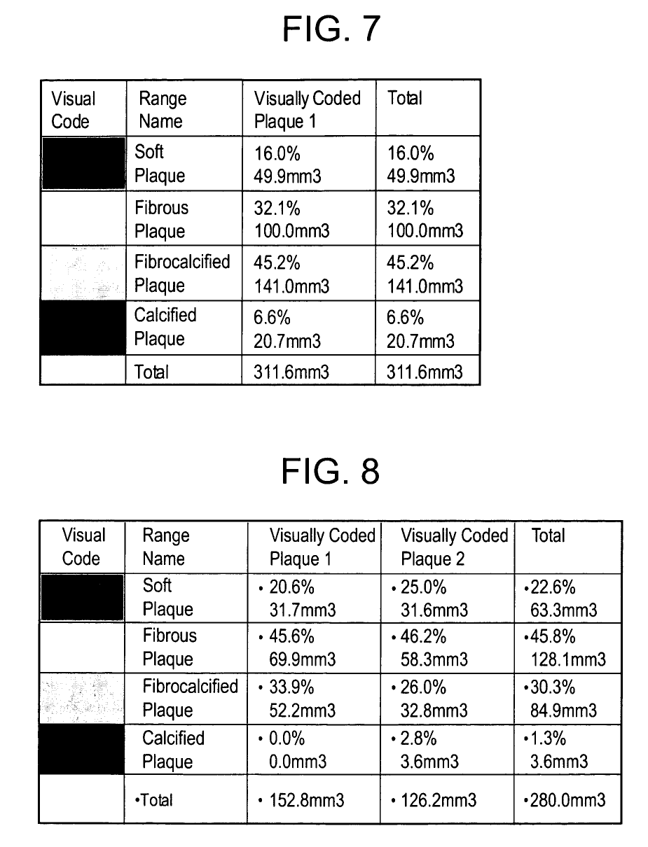

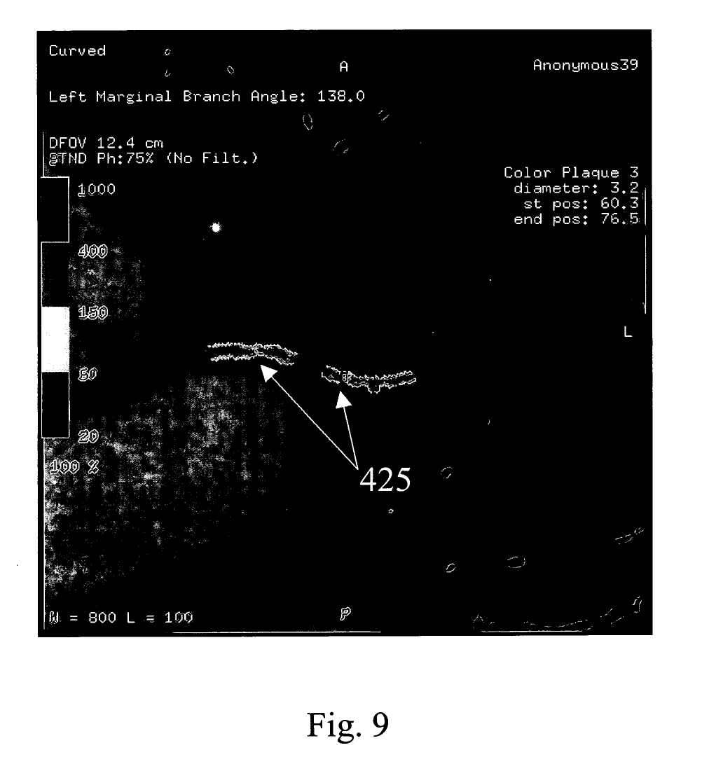

This information is clinically relevant in assessing the burden of soft plaque and other vessel characteristics, and provides both visual and mathematical expressions of the burden. Embodiments of the invention may provide quantitative outputs that will include both volume measurements as well as percent of the entire volume, as depicted in the table of FIG. 7 for a ROI having a single sub-volume, and in FIG. 8 for a ROI having two sub-volumes, which is depicted in the image of FIG. 9 and enumerated by reference numeral 425. FIG. 7 depicts a table showing an example of quantitative output from the feature where “Visually Coded Plaque 1” is the name given to the visual coding deposited with respect to FIG. 6D. FIG. 8 depicts a table showing an example of quantitative output from the feature where “Visually Coded Plaque 1” and “Visually Coded Plaque 2” are the names given to the visual coding deposited with respect to the two regions illustrated in FIG. 9. In FIG. 7, the four visually coded ranges of the ROI have calculated volumes of 49.9 mm³ (cubic millimeters), 100.0 mm³, 141.0 mm³, and 20.7 mm³, respectively. In FIG. 8, there are two visually coded plaques, each with four visually coded ranges relating to the respective sub-volume of the ROI. Armed with this information, various analyses and statistical calculations may be made.

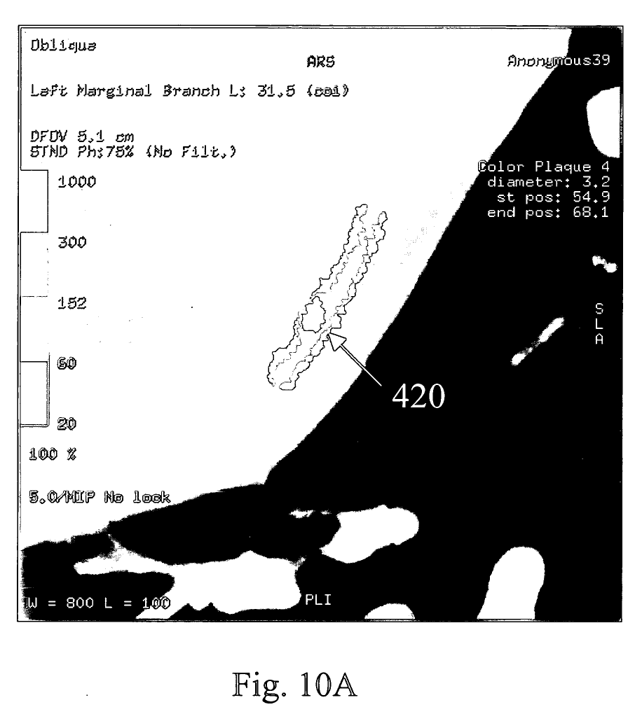

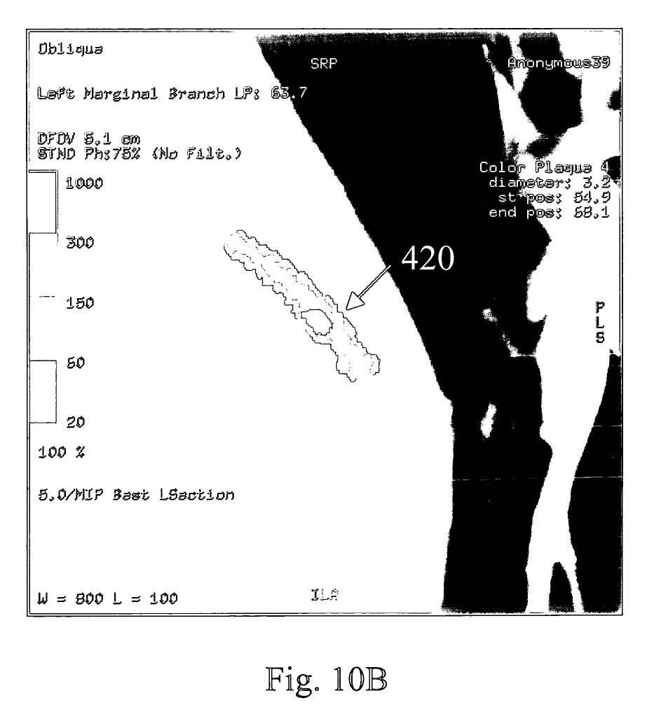

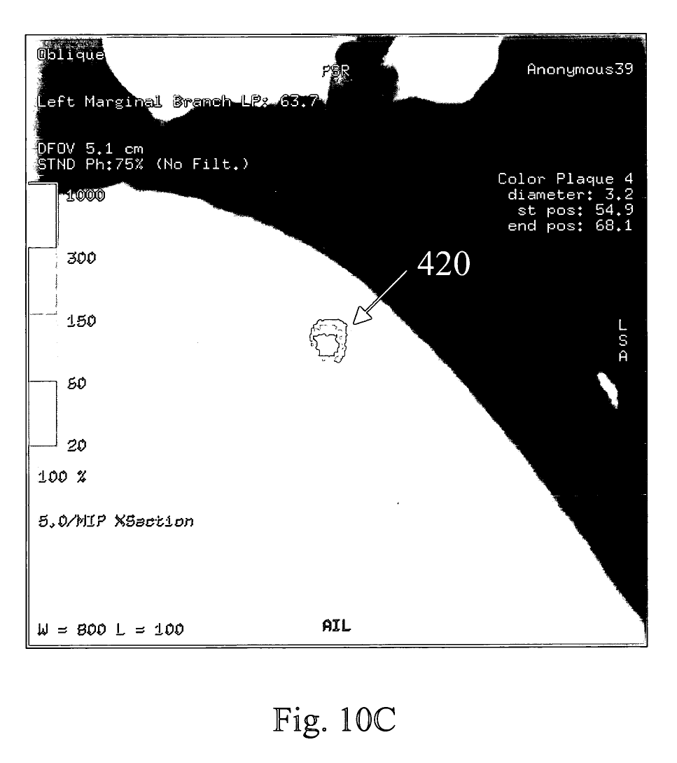

In alternative embodiments, the visual coding may be visualized in other layouts and orientations other than curved reformat view, such as oblique, best L section, and cross section of the vessel, for example, as best seen by referring to FIGS. 10A, B and C, respectively. Additionally, and as previously depicted in FIG. 9, multiple regions may be deposited on a single vessel of a single exam to allow for comparison.

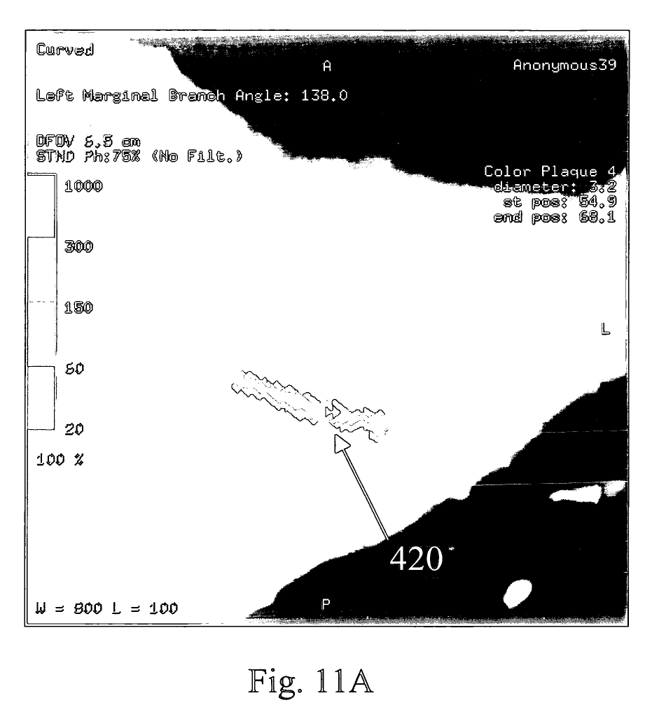

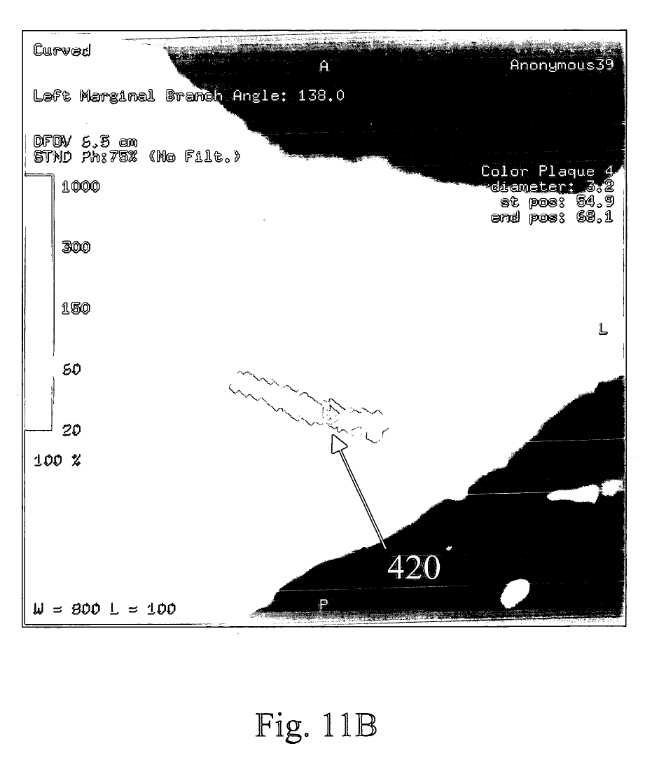

When the visual coding is applied to the vessel, the user may also modify settings in the aforementioned LUT in order to switch from a step mode to a continuous mode visualization, best seen by now referring to FIGS. 11A and B, respectively. The step mode visualization employs a hard line boundary between visually coded regions of the ROI, while the continuous mode visualization employs a soft or transitional boundary between the visually coded regions, which is evident in both the visual coding key at the side of the image and in the visual coding along the vessel itself. Continuous mode is primarily used for visualization and not quantification, and blends the independently colored regions, when employing color coding, by ramping the boundary of consecutive ranges for a smoothed visual effect.

In embodiments of the invention, the user is also able to adjust opacity in order to gain visual assessment of the area and help in the vessel analysis.

Other statistics may be provided inside this ROI, such as minimum, maximum, average and standard deviation of pixel or voxel HU values, as well as histograms displaying the repartition of values among the volume of interest.

While not herein illustrated, it is contemplated that color-coding vessel analysis features may also include the ability for the software to automatically color the entire vessel tracked region along the centerline instead of requiring the user to make the deposit to define the ROI. This enhancement would meet a different use case as it would provide visual inputs for the overall exam but would not isolate specific areas to provide localized (such as ‘proximal LAD’) quantitative results. This is useful to enable fast detection of soft plaque tissue.

It is further contemplated that other embodiments of the invention may encompass: the use of the ROI construction and tissue classification as herein disclosed to analyze other type of tubular structures, such as colon and airways for example; and, the ability to adjust the diameter of the tube locally, thereby making it possible, for example, to better fit the shape of coronaries because this type of vessel decreases from its proximal to its distal part.

Embodiments of the invention may be embodied in the form of computer-implemented processes and apparatuses for practicing those processes. The present invention may also be embodied in the form of a computer program product having computer program code containing instructions embodied in tangible media, such as floppy diskettes, CD-ROMs, hard drives, USB (universal serial bus) drives, or any other computer readable storage medium, wherein, when the computer program code is loaded into and executed by a computer, the computer becomes an apparatus for practicing the invention. The present invention may also be embodied in the form of computer program code, for example, whether stored in a storage medium, loaded into and/or executed by a computer, or transmitted over some transmission medium, such as over electrical wiring or cabling, through fiber optics, or via electromagnetic radiation, wherein when the computer program code is loaded into and executed by a computer, the computer becomes an apparatus for practicing the invention. When implemented on a general-purpose microprocessor as part of imaging system 100, the computer program code segments configure the microprocessor to create specific logic circuits. The technical effect of the executable instructions is the analysis of tissue classes along tubular structures.

As disclosed, some embodiments of the invention may include some of the following advantages: a qualitative fast visual assessment to identify areas of interest for further analysis; a classification of densities into different ranges of interest; a quantitative assessment of volume per each density range over the specified length and diameter; a reproducible preset for performing similar classifications and analysis over various exams; a method that automatically adapts the analyzed region to the shape of the vessel with minimum user interaction; the user's ability to perform quantitative assessment in the vessel area while excluding surrounding tissue by focusing on the tracked vessel; a diagnostic system that provides qualitative and quantitative tissue classification tools based on voxel densities, given the definition by the user of a 3D region of interest (ROI) being a portion of a tubular object; a region of interest that may be a generalized cylinder, that is, a cylinder which “follows” a 3D line that results from a previous vessel tracking process; the ability for the cylinder diameter to be manually adjusted or automatically adjusted according to the diameter of the vessel; the ability for the cylinder diameter to be constant across the section length or vary according to the local diameter of the vessel; the ability to dynamically adjust the start and end positions of the section of the ROI; providing a qualitative fast visual assessment to identify areas of interest for further analysis; providing a classification of densities into different ranges of interest; providing a quantitative assessment of volume per each density range over the specified length and diameter; and, providing a reproducible preset for performing similar classifications and analysis over various exams.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

1. A method for analyzing tissue classes along a tubular structure defined by voxels, comprising:
constructing a tubular shaped region of interest (ROI) along a predetermined centerline of the tubular structure according to the following:
defining at least one point along the centerline to define the extremities of the ROI;
defining and/or computing a diameter corresponding to the maximum of the diameters of the orthogonal sections of the ROI;
applying a plurality of contiguous unit volumes along the centerline between the extremities of the ROI, the unit volumes having an overall dimension equal to or less than the maximum of the diameters of the orthogonal sections of the ROI;
computing a first volume by the union of the unit volumes; and
defining a final volume of the ROI as the connex part of the first volume that contains the middle of the tubular structure; and
analyzing the final volume with respect to tissue classes present therein.

2. The method of claim 1, wherein the constructing further comprises:
modifying the extremities of the first volume by subtracting two volumes from the first volume, one subtracted volume being subtracted from each extremity, thereby establishing flat surfaces at the extremities of the first volume.

3. The method of claim 1, wherein:
the unit volumes comprise spheres.

4. The method of claim 1, wherein:
the unit volumes comprise cylinders.

5. The method of claim 1, further comprising:
classifying the voxels in the ROI according to a visual coding scheme associated with the Hounsfield Unit (HU) values of the voxels.

6. The method of claim 5, wherein the analyzing comprises:
analyzing the visually coded ROI with respect to a classification of tissue densities associated with the visual coding, each tissue density classification having a density range correlating with the visual coding.

7. The method of claim 6, further comprising:
analyzing the visually coded ROI with respect to the volume of each density range.

8. The method of claim 7, further comprising:
analyzing the visually coded ROI with respect to a statistical analysis of each density range.

9. The method of claim 5, wherein:
the visual coding scheme comprises a color coding scheme.

10. The method of claim 5, wherein:
the visual coding scheme comprises a discrete gray scale coding scheme.

11. The method of claim 5, wherein:
the visual coding scheme distinguishes the ROI from surround tissue displayed in grayscale.

12. The method of claim 1, wherein:
the diameter corresponding to the maximum of the diameters of the orthogonal sections of the ROI varies according to the local diameter of the vessel.

13. The method of claim 1, wherein the analyzing comprises:
analyzing the final volume by employing both visual and mathematical expressions of the burden of vessel deposit present in the ROI.

14. The method of claim 1, wherein:
the ROI comprises a first sub-volume and a second sub-volume.

15. The method of claim 5, wherein:
the visual coding is visualized in a layout and orientation comprising curved reformat view, oblique view, best L section view, cross section view, or any combination comprising at least one of the foregoing views.

16. The method of claim 5, wherein:
the visual coding is switchable from a step mode to a continuous mode visualization.

17. The method of claim 5, wherein:
the opacity of the visual coding is adjustable.

18. An apparatus for acquiring tissue images and analyzing tissue classes along tubular structures, the apparatus comprising:
a medical scanner for generating a volume of image data relating to a region of interest;
a data acquisition system for acquiring the volume of image data;
an image reconstructor for reconstructing a viewable image from the volume of image data;
a database for storing information from the data acquisition system and the image reconstructor;
an operator interface for managing the medical scanner, the data acquisition system, the image reconstructor, the database, or any combination comprising at least one of the foregoing;
a computer for analyzing the reconstructed volume of image data and displaying the viewable image, the computer being responsive to said operator interface; and
a storage medium, readable by a processing circuit, storing instructions for execution by the processing circuit for:
constructing a tubular shaped region of interest (ROI) along a predetermined centerline of the tubular structure according to the following:
responding to at least one defined point along the centerline to define the extremities of the ROI;
computing a diameter corresponding to the maximum of the diameters of the orthogonal sections of the ROI;
applying a plurality of contiguous unit volumes along the centerline between the. extremities of the ROI, the unit volumes having an overall dimension equal to or less than the maximum of the diameters of the orthogonal sections of the ROI;
computing a first volume by the union of the unit volumes; and
computing a final volume of the ROI as the connex part of the first volume that contains the middle of the tubular structure; and
analyzing the final volume with respect to tissue classes present therein.

19. A computer program product embodied in a tangible medium for analyzing tissue classes along tubular structures, the product comprising computer readable instructions for:
constructing a tubular shaped region of interest (ROI) along a predetermined centerline of the tubular structure according to the following:
responding to at least one defined point along the centerline to define the extremities of the ROI;
computing a diameter corresponding to the maximum of the diameters of the orthogonal sections of the ROI;
applying a plurality of contiguous unit volumes along the centerline between the extremities of the ROI, the unit volumes having an overall dimension equal to or less than the maximum of the diameters of the orthogonal sections of the ROI;
computing a first volume by the union of the unit volumes; and
computing a final volume of the ROI as the connex part of the first volume that contains the middle of the tubular structure; and
analyzing the final volume with respect to tissue classes present therein.
